(19) United States
(12) Patent Application Publication
(10) Pub. No.: US 2026/0263839 A1
(43) Pub. Date: Sep. 10, 2026

(54) OPTIMAL BERYLLIUM TARGET INTERNAL COOLING STRUCTURE FOR HIGH HEAT FLUX REMOVAL FOR A-BNCT

(71) Applicant: DAWONMEDAX,. LTD., Ansan-si Gyeonggi-do (KR)

(72) Inventors: Young Soon BAE, Incheon (KR); Dong Su KIM, Siheung-si, Gyeonggi-do (KR); Myung Heon LEE, Incheon (KR); Na Hyoung PARK, Incheon (KR); Sun Soon PARK, Suwon-si, Gyeonggi-do (KR)

(73) Assignee: DAWONMEDAX,. LTD., Ansan-si Gyeonggi-do (KR)

(21) Appl. No.: 19/165,032

(22) PCT Filed: Mar. 28, 2023
(86) PCT No.: PCT/KR2023/004069

§ 371 Date: Jun. 1, 2026

(30) Foreign Application Priority Data
Mar. 13, 2023 (KR) 10-2023-0032609

(51) Int. Cl. A61N 5/10; A61N 5/00

(52) U.S. Cl. CPC A61N 5/1077; A61N 2005/002; A61N 2005/109

(57) ABSTRACT

An optimal beryllium target internal cooling structure for high heat flux removal for A-BNCT (Accelerator-based Boron Neutron Capture Therapy) is disclosed. The cooling structure according to an embodiment of the present disclosure includes a beam shaping assembly, a plurality of beam ducts, some of which are vertically inserted into the beam shaping assembly, and extended and combined along a path of a proton beam, a target unit disposed at one end of the plurality of beam ducts and generating neutrons by colliding with the proton beam, and a cooling line continuously penetrated and formed inside the plurality of beam ducts and through which cooling fluid passes.

### CROSS-REFERENCE TO RELATED PATENT APPLICATIONS

The present application is based upon and claims priority to Korean Patent Application No. 10-2023-0032609 filed on Mar. 13, 2023. The contents of the foregoing application are incorporated herein by reference in its entirety.

### TECHNICAL FIELD

The present disclosure relates to an optimal beryllium target internal cooling structure for high heat flux removal of accelerator-based boron neutron capture therapy (hereinafter referred to as “A-BNCT”) using neutrons.

### BACKGROUND OF THE INVENTION

The description in this section merely provides background information related to the present disclosure and does not necessarily constitute the related art.

Conventional radiation therapy includes X-ray therapy, proton therapy, and heavy particle therapy depending on the radiation used. Unlike conventional radiation therapy, in the case of A-BNCT, radiation exposure minimization of normal tissues, cell-unit treatment of cancer tissues, and treatment of internal radiation cancer such as external ear cancer, head and neck cancer and malignant skin cancer are possible. In addition, there are benefits in terms of economic aspects such as low-cost facility construction cost, reduction in the number of medical procedures, and low cost of treatment.

The A-BNCT is configured of an injector, a Radio Frequency Quadrupole (RFQ), and a Drift Tube Linear Accelerator (DTL). The proton beam made through the injector, which is an ion source, is focused and accelerated by four electrodes through the RFQ. Thereafter, it is accelerated again through the DTL and irradiates a beam with a neutron generator. Accordingly, a neutron beam, which is the final output of an accelerator, is irradiated to the affected area of a patient, and nuclear fission occurs in cancer cells combined with boron to kill only the cancer cells without destroying normal cells, thereby performing treatment.

The A-BNCT may generate high-intensity epithermal neutron flux with a treatment depth of, for example, 7-8 cm, which is deeper than 2-3 cm of thermal neutrons. To form the epithermal neutron flux, a target including a beryllium layer may be used. However, when the beryllium target is continuously irradiated with protons, a blistering phenomenon may occur due to an increase in pressure of hydrogen vapor captured inside the beryllium target. This blistering phenomenon has an issue of reducing the lifetime of the beryllium target.

Furthermore, when the beryllium target is irradiated with proton beam energy and operation and stopping are repeated, the temperature of the beryllium target may rise and fall rapidly. Due to this repetition of temperature rise and fall, the beryllium target may be deformed due to thermal expansion and contraction (Thermal Cycling), and a gap may occur in a vacuum seal that maintains a vacuum, which may be an obstacle element to continuous operation of an A-BNCT device.

The A-BNCT, based on an accelerator, generates neutrons through a nuclear reaction by a collision between a target material and accelerated particles. The neutrons generated in the nuclear reaction spread uniformly in all directions based on the center of mass coordinate system.

Since the generated neutrons are of higher energy than necessary for immediate use by a patient and a large amount of gamma rays are generated in addition to neutrons, neutrons may not be immediately used near a target. Accordingly, a beam shaping assembly serves to slow the generated neutrons into epithermal neutrons having an appropriate energy level and to partially remove harmful radiation that is exposed to a patient.

The target where neutrons are generated and the position where a patient is irradiated with neutron rays have a predetermined distance due to the beam shaping assembly. Because neutrons spread out in all directions, a significant portion of the neutrons reaching a patient are lost. The A-BNCT according to the related art dissipates heat by installing a separate cooling line for high heat flux caused by the collision between the target material and the accelerated particles. However, there is an issue in that the loss rate of neutrons reaching the affected area increases due to an air gap between the target and the beam shaping assembly due to equipment such as a cooling line.

### SUMMARY OF THE INVENTION

An optimal beryllium target internal cooling structure for high heat flux removal for A-BNCT according to an embodiment forms a cooling channel and a plurality of protrusions inside a target unit to convert cooling fluid into a turbulent flow state, so that the high heat flux generated inside the target unit may be effectively reduced.

The optimal beryllium target internal cooling structure for high heat flux removal for A-BNCT according to an embodiment may reduce damage to a beryllium sheet from a proton beam.

In the optimal beryllium target internal cooling structure for high heat flux removal for A-BNCT according to an embodiment, two cooling lines are continuously formed inside a plurality of beam ducts, so that the air gap between the target unit and a beam shaping assembly may be minimized.

The optimal beryllium target internal cooling structure for high heat flux removal for A-BNCT according to an embodiment may use a cooling chamber formed by combining a first target unit and a second target unit to evenly distribute the cooling fluid moving inside the cooling channel.

The optimal beryllium target internal cooling structure for high heat flux removal for A-BNCT according to an embodiment may uniformly supply the cooling fluid to the target unit by using the cooling channel formed between the first target unit and the second target unit.

The optimal beryllium target internal cooling structure for high heat flux removal for A-BNCT according to an embodiment is easy to assemble and maintainability may be improved by combination and disassembly among a first beam duct, a second beam duct, a third beam duct, a fourth beam duct, and a fifth beam duct using bolts and nuts.

The aspects of the present disclosure are not limited to those mentioned above, and other aspects not mentioned herein will be clearly understood by those skilled in the art from the following description.

According to an embodiment of the present disclosure, there is provided an optimal beryllium target internal cooling structure for high heat flux removal for A-BNCT (Accelerator-based Boron Neutron Capture Therapy), the cooling structure including: a beam shaping assembly; a plurality of beam ducts, some of which are vertically inserted into the beam shaping assembly, and extended and combined along a path of a proton beam; a target unit disposed at one end of the plurality of beam ducts and generating neutrons by colliding with the proton beam; and a cooling line continuously penetrated and formed inside the plurality of beam ducts and through which cooling fluid passes.

According to an embodiment, an optimal beryllium target internal cooling structure for high heat flux removal for A-BNCT forms a cooling channel and a plurality of protrusions inside a target unit to convert cooling fluid into a turbulent flow state, so that the high heat flux generated inside the target unit may be effectively reduced.

According to an embodiment, the optimal beryllium target internal cooling structure for high heat flux removal for A-BNCT may reduce damage to a beryllium sheet from a proton beam.

According to an embodiment, in the optimal beryllium target internal cooling structure for high heat flux removal for A-BNCT, two cooling lines are continuously formed inside a plurality of beam ducts, so that the air gap between the target unit and a beam shaping assembly may be minimized.

According to an embodiment, the optimal beryllium target internal cooling structure for high heat flux removal for A-BNCT may use a cooling chamber formed by combining a first target unit and a second target unit to evenly distribute the cooling fluid moving inside the cooling channel.

According to an embodiment, the optimal beryllium target internal cooling structure for high heat flux removal for A-BNCT may uniformly supply the cooling fluid to the target unit by using the cooling channel formed between the first target unit and the second target unit.

According to an embodiment, the optimal beryllium target internal cooling structure for high heat flux removal for A-BNCT is easy to assemble, and maintainability may be improved by combination and disassembly among a first beam duct, a second beam duct, a third beam duct, a fourth beam duct, and a fifth beam duct using bolts and nuts.

### BRIEF DESCRIPTION OF THE DRAWINGS

FIG. 1 is a view of the overall configuration of a beam shaping assembly and an A-BNCT target system assembly according to an embodiment of the present disclosure.

FIG. 2 is a cross-sectional view of a combination relationship between the beam shaping assembly and the A-BNCT target system assembly of FIG. 1.

FIG. 3 is a perspective view of the A-BNCT target system assembly according to an embodiment of the present disclosure.

FIG. 4 is a cross-sectional view taken along line A-A of FIG. 3.

FIG. 5 is an exploded perspective view of a target unit and a first beam chamber according to an embodiment of the present disclosure.

FIG. 6 is an exploded perspective view of the target unit viewed from the rear according to an embodiment of the present disclosure.

FIG. 7 is a right cross-sectional view taken along line B-B of FIG. 3.

### DETAILED DESCRIPTION OF THE INVENTION

Hereinafter, some embodiments of the disclosure will be described in detail with reference to exemplary drawings. It should be noted that, in assigning reference numerals to the components in each drawing, the same components have the same numerals as much as possible, even if they are shown in different drawings. In addition, in describing the disclosure, when it is determined that a specific description of a related known configuration or function may obscure the gist of the disclosure, a detailed description thereof will be omitted.

In describing the components of the present invention, terms such as “first”, “second”, “A”, “B”, “(a)”, “(b)” and the like may be used. These terms are merely used to distinguish one component from another component, and the nature, sequence, or order of that component is not limited by the terms. Throughout the specification, when a portion is referred to as “comprising” or “including” a component, this means that other components may be further included instead of excluding other components unless otherwise stated.

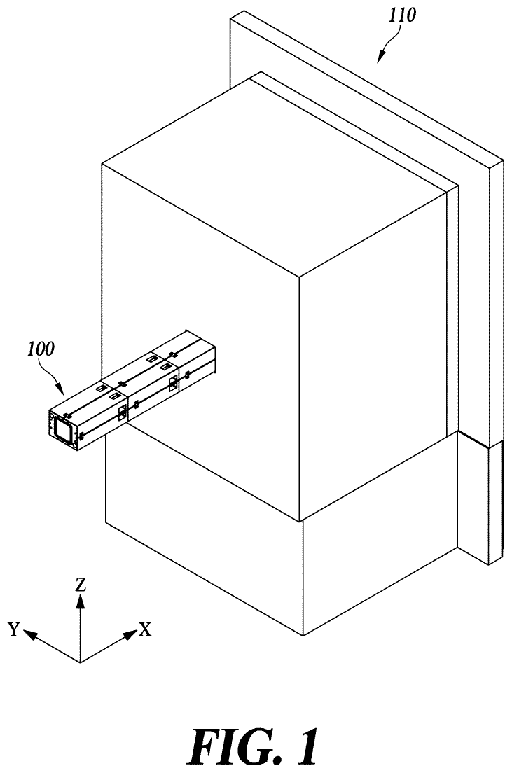

FIG. 1 is a view of the overall configuration of a beam shaping assembly and an A-BNCT target system assembly according to an embodiment of the present disclosure.

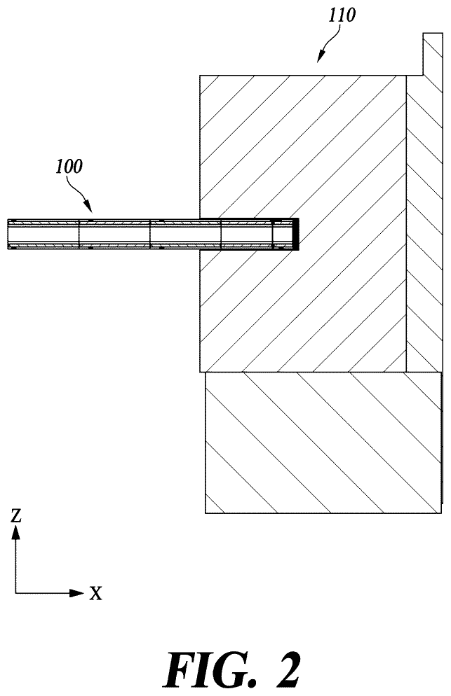

FIG. 2 is a cross-sectional view of a combination relationship between the beam shaping assembly and the A-BNCT target system assembly of FIG. 1.

Referring to FIGS. 1 and 2, an A-BNCT target system assembly 100 may be combined with a beam shaping assembly 110. The A-BNCT target system assembly 100 maintains a fixed state. The beam shaping assembly 110 may be separated from the A-BNCT target system assembly 100 by being slid in an x-axis direction. A patient may be positioned in front of the beam shaping assembly 110 in an x-axis direction by a predetermined distance.

Since the speed of neutrons is reduced due to the beam shaping assembly 110, when the neutrons pass through a target and reach the affected area of a patient, in order for a large amount of neutrons to reach the affected area, the air gap between the A-BNCT target system assembly 100 and the beam shaping assembly 110 needs to be minimized. The amount of neutrons delivered to the affected area of a patient increases as the air gap between the A-BNCT target system assembly 100 and the beam shaping assembly 110 is minimized.

The beam shaping assembly 110 may include a moderator (not shown), a collimator (not shown), a neutron reflector (not shown), and a shield (not shown).

The moderator serves to slow down the neutrons generated on a target and en route to an exit to an appropriate energy level. The moderator may be a disc-shaped member made of lead, iron, aluminum, or calcium fluoride stacked in a direction in which epithermal neutrons are emitted from the target to slow down neutron rays generated from the target.

The collimator gathers neutrons towards the treatment site of a patient. The beam shaping assembly 110 is preferably configured to improve the flux and forward directional properties of the epithermal neutrons.

The neutron reflector serves to shield epithermal neutrons, gamma rays, and fast neutrons generated from a target and leaking to the outside of equipment. The neutron reflector may be configured of Pb (plumbum), and by being disposed while surrounding the target and the moderator in an annular shape, the loss of epithermal neutron rays may be prevented by preventing the escape of the epithermal neutrons.

A proton beam with a high heat flux accelerated from the A-BNCT target system assembly 100 is incident on a target to generate neutrons. The generated neutrons are decelerated to epithermal neutrons having an appropriate energy level by the beam shaping assembly 110 and are incident on the affected area of a patient located in the front thereof.

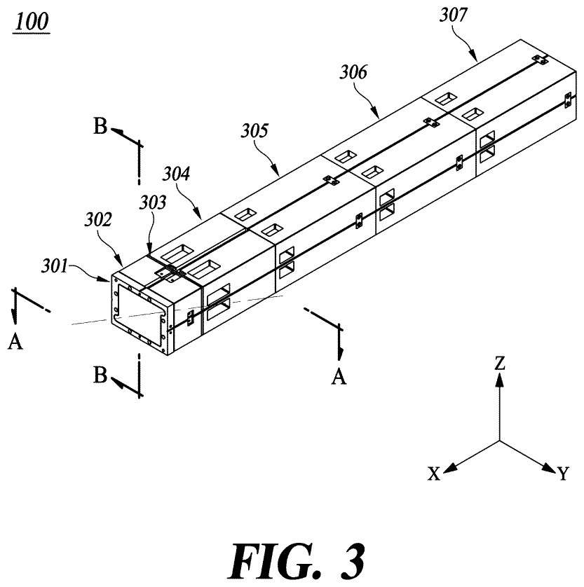

FIG. 3 is a perspective view of the A-BNCT target system assembly according to an embodiment of the present disclosure.

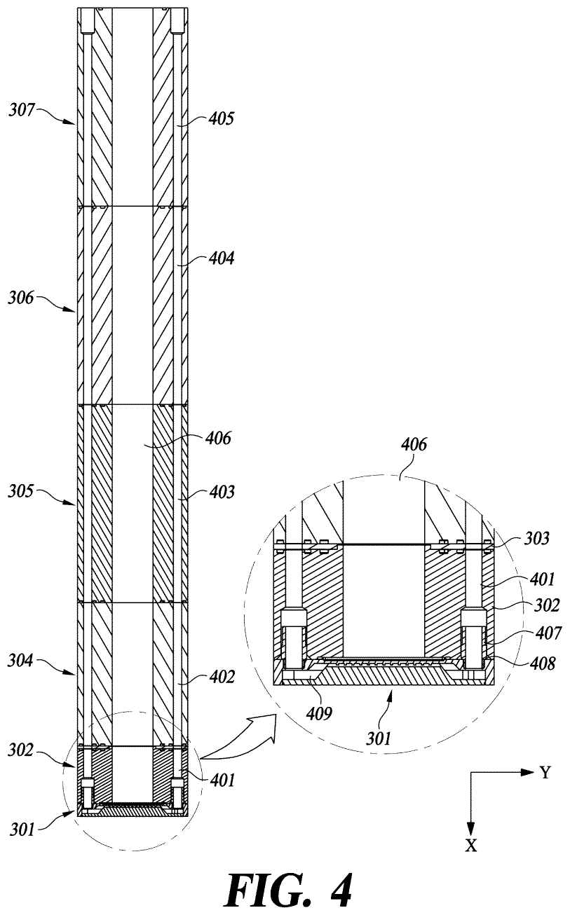

FIG. 4 is a cross-sectional view taken along line A-A of FIG. 3.

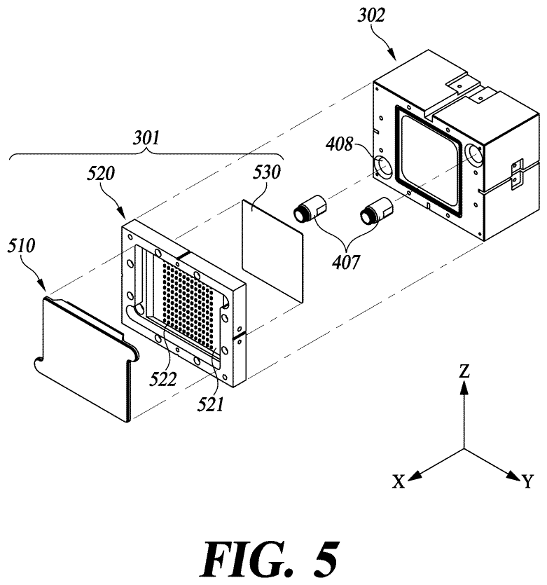

FIG. 5 is an exploded perspective view of a target unit and a first beam chamber according to an embodiment of the present disclosure.

Referring to FIGS. 3 to 5, the A-BNCT target system assembly 100 includes all or part of a target unit 301, a first beam duct 302, an insulation member 303, a second beam duct 304, a third beam duct 305, a fourth beam duct 306, and a fifth beam duct 307.

The target unit 301 is disposed at one end of the A-BNCT target system assembly 100. In the target unit 301, protons moving along a proton beam hollow tube 406 formed by the first beam duct 302 to the fifth beam duct 307 collide with each other or one another. Herein, the proton beam hollow tube 406 may be a rectangular hollow tube. Each corner of the proton beam hollow tube may be chamfered.

The target unit 301 includes a first target unit 510, a second target unit 520, and a beryllium sheet 530. Herein, the beryllium sheet 530 is a beryllium target (Be-Cu conjugant).

The first target unit 510 and the second target unit 520 may be formed based on a copper material. The first target unit 510 and the second target unit 520 may be a heat sink dissipating a high heat flux of the beryllium sheet 530.

The second target unit 520 includes a combination unit 521 combined with the first target unit 510. The combination unit 521 includes a plurality of protrusions 522. The plurality of protrusions 522 are formed on one surface of the combination unit 521 and project in the direction of the first target unit 510 (x-axis direction). The plurality of protrusions 522 are arranged in the combination unit 521 based on a predetermined pattern. When the plurality of protrusions 522 are arranged in the combination unit 521, it is preferable that the plurality of protrusions 522 be arranged in an area corresponding to the cross-sectional area of the beryllium sheet 53. For example, when the horizontal and vertical lengths of the beryllium sheet 530 are 10 cm, the number of the plurality of protrusions 522 may be 169.

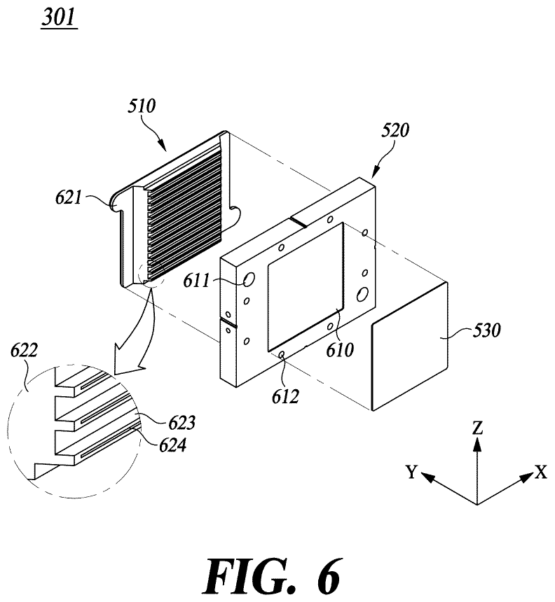

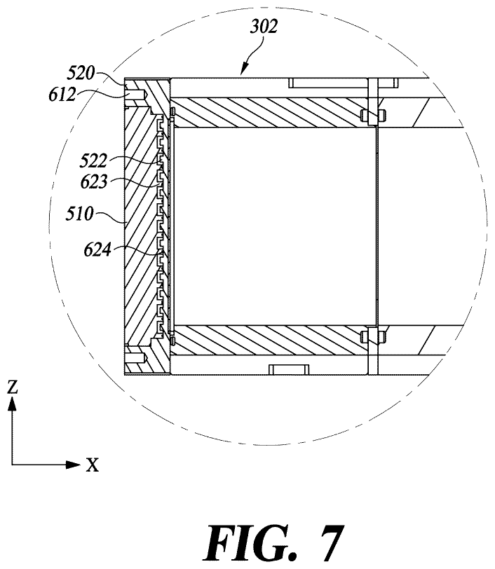

According to an embodiment of the present disclosure, the plurality of protrusions 522 may be rectangular-shaped protrusions. However, the shape of the plurality of protrusions 522 is not limited thereto. The plurality of protrusions 522 change the flow of cooling fluid flowing inside the target unit 301. Herein, the cooling fluid may be, for example, cooling water. Details of the movement of the cooling fluid inside the target unit 301 will be described with reference to FIGS. 6 and 7.

The cross-sectional shape of the second target unit 520 corresponds to the cross-sectional shape of the first beam duct 302.

The beryllium sheet 530 is combined with the rear of the second target unit 520. The horizontal and vertical lengths of the beryllium sheet 530 may be, for example, 10 cm and 10 cm.

The beryllium sheet 530 is disposed at one end of the proton beam hollow tube 406. The beryllium sheet 530 may shield one end of the proton beam hollow tube 406. The beryllium sheet 530 may be irradiated with the proton beam moving inside the proton beam hollow tube 406, and beryllium epithermal neutrons may be radiated based on impact energy between the proton beam and the beryllium sheet 530.

The first target unit 510 is combined with the second target unit 520. The first target unit 510 has a predetermined cross-sectional shape to be combined with the second target unit 520. For example, it is preferable that the cross-sectional shape of the first target unit 510 is formed smaller than the cross-sectional area of the second target unit 520, and is formed to include all of the plurality of protrusions 522 formed on the combination unit 521 of the second target unit 520. In addition, the cross-sectional shape of the first target unit 510 may include a projection 621 partially projecting in a y-axis direction and a-y-axis direction so that the cooling fluid introduced from a cooling port 407 combined with the second target unit 520 is guided into a cooling chamber 409.

According to an embodiment of the present disclosure, the first target unit 510 and the second target unit 520 are combined, and the cross-sectional shape of the first target unit 510 is determined to form the manifold-shaped cooling chamber 409 at both sides inside the target unit 301.

The insulation member 303 may be, for example, a plate made of polyethylene (PE). The insulation member 303 is disposed between the first beam duct 302 and the second beam duct 304. The insulation member 303 electrically separates the first beam duct 302 and the second beam duct 304.

In order to estimate a proton current value, resistors (not shown) are respectively connected to the first beam duct 302 and the second beam duct 304. Accordingly, the proton current value may be estimated based on the input voltage value and the respective resistance values connected to the first beam duct 302 and the second beam duct 304. Herein, the proton current value is used to evaluate the performance of the A-BNCT target system assembly 100 and the performance of the neutron irradiation system. For example, when the proton current value is maintained above a predetermined value, the amount of neutrons produced increases, and the performance of the neutron irradiation system capable of proper irradiation is guaranteed. Accordingly, the proton current value in the A-BNCT may be a factor for maximizing the treatment effect and evaluating the performance of the irradiation system and the neutron generation.

The A-BNCT target system assembly 100 further includes the first beam duct 302, the second beam duct 304, the third beam duct 305, the fourth beam duct 306, and the fifth beam duct 307.

The first beam duct 302, the second beam duct 304, the third beam duct 305, the fourth beam duct 306, and the fifth beam duct 307 may be formed based on an aluminum material.

The first beam duct 302, the second beam duct 304, the third beam duct 305, the fourth beam duct 306, and the fifth beam duct 307 may be combined with each other or one another using bolts and nuts. A plurality of beam ducts 302, 304, 305, 306, and 307 is formed with at least one sealing O-ring groove for vacuum respectively on combination surfaces combined with each other.

The first beam duct 302 includes two sealing O-ring grooves 408 for cooling. The sealing O-ring groove 408 for cooling is formed at one end of a first cooling line 401.

The first beam duct 302 includes the first cooling line 401. The second beam duct 304 includes a second cooling line 402. The third beam duct 305 includes a third cooling line 403. The fourth beam duct 306 includes a fourth cooling line 404. The fifth beam duct 307 includes a fifth cooling line 405. The first cooling line 401 to the fifth cooling line 405 are continuously disposed. Accordingly, the first cooling line 401 to the fifth cooling line 405 form two continuous cooling lines. Herein, the first cooling line 401 to the fifth cooling line 405 form an inflow cooling line and a recovery cooling line. The inflow cooling line is formed so that cooling fluid moves from the rear of the A-BNCT target system assembly 100 to the target unit 301. The recovery cooling line is formed so that the cooling fluid moves from the target unit 301 to the rear of the A-BNCT target system assembly 100. The inflow cooling line and the recovery cooling line are formed continuously and cross-symmetrically based on the cross sections of the respective beam ducts 302, 304, 305, 306, and 307.

The first cooling line 401 to the fifth cooling line 405 are connected in an x-axis direction to form one cooling line. The first cooling line 401 to the fifth cooling line 405 each may be two or more. Accordingly, the first cooling line 401 to the fifth cooling line 405 may form two cooling lines through which cooling fluid is introduced or discharged. According to an embodiment of the present disclosure, the first cooling line 401 to the fifth cooling line 405 may be formed in a cross-symmetrical manner inside the first beam duct 302 to the fifth beam duct 307.

Accordingly, when the A-BNCT target system assembly 100 is inserted into the beam shaping assembly 110 because the cooling line is not formed outside and is formed inside the plurality of beam ducts 302, 304, 305, 306, and 307, the A-BNCT target system assembly 100 may be compactly combined. Accordingly, an air gap between the combination surfaces of the A-BNCT target system assembly 100 and the beam shaping assembly 110 may be minimized.

The cooling port 407 is combined with one end of the first cooling line 401. Herein, a portion of the first cooling line 401 may have a partially increased diameter to accommodate the cooling port 407. When the cooling port 407 is combined with one end of the first cooling line 401, the cooling port 407 may be braze-combined.

FIG. 6 is an exploded perspective view of the target unit viewed from the rear according to an embodiment of the present disclosure.

FIG. 7 is a right cross-sectional view taken along line B-B of FIG. 3.

Referring to FIGS. 6 and 7, the first target unit 510 partially projects in the direction of the second target unit 520 (-x axis direction). Herein, an inclined surface 622 may be a tapered surface. The taper angle of the inclined surface 622 may be, for example, 130°. A cooling channel 623 extending in a y-axis direction is formed at the other end of the inclined surface 622. The cooling channel 623 is formed so that the cooling fluid introduced from the first cooling line 401 uniformly flows inside the target unit 301. The cooling channel 623 may be one flow path formed by combining the first target unit 510 and the second target unit 520. A plurality of cooling channels 623 may be formed on the first target unit 510. The cooling channel 523 is formed on one surface of the first target unit 510 in the direction of the second target unit 520 (−x axis direction). The number of cooling channels 623 is preferably formed to correspond to the size of the beryllium sheet 530. For example, when the horizontal and vertical lengths of the beryllium sheet 530 are 10 cm, the number of cooling channels 623 may be 13.

The plurality of protrusions 522 formed on the combination unit 521 of the second target unit 520 are arranged inside the cooling channel 623. Accordingly, the cooling fluid introduced from the first cooling line 401 is guided in a-z direction by the cooling chamber 409. The cooling fluid in the cooling chamber 409 uniformly flows into the cooling channel 623, and collides with the plurality of protrusions 522 formed in the cooling channel 623 to form a turbulent flow. The cooling fluid in a turbulent flow state may effectively dissipate the high heat flux of the beryllium sheet 530. The cooling fluid in the turbulent flow state increases the local heat transfer. In the cooling fluid in the turbulent flow state, continuous exchange occurs among fluid particles while the flow velocity inside the fluid changes irregularly. Due to this exchange, the heat inside the cooling fluid is evenly distributed. In addition, since the heat transfer area of the cooling fluid in the turbulent flow state is larger than that of the cooling fluid in a laminar flow state, the heat dissipation benefit is superior. Accordingly, the cooling fluid passing through the inside of the cooling channel 623 collides with the plurality of protrusions 522 to generate a turbulent flow, so that the high heat flux of the beryllium sheet 530 may be effectively dissipated.

The cooling channel 623 includes a filler groove 624. A brazing filler is inserted into the filler groove 624 so that the first target unit 510 and the second target unit 520 may be completely bonded. The filler groove 624 is formed so that the cooling fluid moving inside the cooling channel 623 does not leak to the outside.

The second target unit 520 includes a fastening groove 611 combined with the cooling port 407. The fastening groove 611 is formed on the second target unit 520 according to the location of the cooling port 407. The fastening groove 611 may have a screw thread formed therein so as to be combined with a portion of the cooling port 407. In addition, the second target unit 520 includes a plurality of tapping holes 612. Tapping screws may be combined with the plurality of tapping holes 612 so that the target unit 301 is combined with the first beam duct 302.

The second target unit 520 includes a seating surface 610 for accommodating the beryllium sheet 530. The size of the seating surface 610 corresponds to the size of the beryllium sheet 530. The depth of the seating surface 610 corresponds to the thickness of the beryllium sheet 530. The beryllium sheet 530 may be braze-bonded to the seating surface 610.

The above description is merely illustrative of the technical idea of the present embodiment, and various modifications and variations will be possible to those skilled in the art without departing from the essential characteristics of the present embodiment. Therefore, the present embodiments are not intended to limit but to explain the technical idea of the present embodiment, and the scope of the technical idea of this embodiment is not limited by this embodiment. The protection scope of the present embodiment should be interpreted by the following claims, and all technical ideas falling within the scope equivalent thereto should be interpreted as being included in the scope of rights of the present embodiment.

1. An optimal beryllium target internal cooling structure for high heat flux removal for A-BNCT (Accelerator-based Boron Neutron Capture Therapy), the cooling structure including:
a beam shaping assembly;
a plurality of beam ducts, some of which are vertically inserted into the beam shaping assembly, and extended and combined along a path of a proton beam;
a target unit disposed at one end of the plurality of beam ducts and generating neutrons by colliding with the proton beam; and
a cooling line continuously penetrated and formed inside the plurality of beam ducts and through which cooling fluid passes.

2. The cooling structure of claim 1, wherein the cooling line includes:
an inflow cooling line through which the cooling fluid flows into the target unit; and
a recovery cooling line through which the cooling fluid moving inside the target unit is recovered,
wherein the inflow cooling line and the recovery cooling line are formed in a cross-symmetrical manner based on the plurality of beam ducts.

3. The cooling structure of claim 1, wherein the target unit includes:
a first target unit including a cooling channel formed along a y-axis direction so that the cooling fluid introduced through the cooling line passes through an inside of the target unit uniformly;
a second target unit formed based on a cross-sectional area corresponding to the beam duct and including a combination unit combined with the first target unit; and
a beryllium sheet braze-combined with a seating surface formed on one surface of the second target unit.

4. The cooling structure of claim 3, wherein the second target unit further includes a fastening groove fastened with a cooling port extended to and combined with one end of the cooling line, and
the cooling port is formed based on a cylindrical shape corresponding to a cross-sectional area of the cooling line.

5. The cooling structure of claim 3, wherein the combination unit is formed based on a predetermined arrangement pattern of a plurality of protrusions accommodated in the cooling channel.

6. The cooling structure of claim 3, wherein the cooling channel includes a filler groove into which a brazing filler is inserted along a longitudinal direction.

7. The cooling structure of claim 3, wherein the first target unit is braze-combined with the second target unit to form a manifold shape on both inner sides, and guides the cooling fluid in z and −z directions to form a cooling chamber supplying the cooling fluid to an inside of the cooling channel.

8. The cooling structure of claim 1, wherein the plurality of beam ducts includes:
a first beam duct combined with the target unit; and
a second beam duct to a fifth beam duct extended and combined based on the first beam duct,
wherein an insulation member is disposed between the first beam duct and the second beam duct.

9. The cooling structure of claim 8, wherein the insulation member electrically separates the first beam duct and the second beam duct.

10. The cooling structure of claim 8, wherein the first beam duct is formed with a sealing O-ring groove for cooling that corresponds to a location of the cooling line so as to prevent leakage of the cooling fluid.

11. The cooling structure of claim 8, wherein the plurality of beam ducts is formed with at least one sealing O-ring groove for vacuum respectively on combination surfaces combined with each other.
